(19) United States
(12) Patent Application Publication
(10) Pub. No.: US 2005/0186286 A1
(43) Pub. Date: Aug. 25, 2005

(54) Skin decellularization method, acellular dermal matrix and production method therefore employing said decellularization method, and composite cultured skin employing said matrix

(76) Inventor: Yoshihiro Takami, Chofu-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/785,090

(22) Filed: Feb. 25, 2004

(51) Int. Cl. A61K 35/12; C12P 21/06

(52) U.S. Cl. 424/572; 435/68.1

(57) ABSTRACT

A method for decellularizing separated skin is provided, the method involving simultaneous treatment with a protease and a surfactant. A highly reliable acellular dermal matrix and an optimum allogeneic acellular dermal matrix for simultaneous grafting with an autologous skin graft are also provided. Furthermore, there is provided a method for producing an acellular dermal matrix, the method including a decellularizing step of treating separated skin simultaneously with a protease and a surfactant, and there is also provided a composite cultured skin employing as a substrate the acellular dermal matrix.

### BACKGROUND OF THE INVENTION

1. Field of the Invention

The present invention relates to a method for decellularizing separated skin, an acellular dermal matrix obtained by the decellularization method, a method for producing an acellular dermal matrix utilizing the decellularization method, and a composite cultured skin employing the acellular dermal matrix as a substrate.

2. Description of the Related Art

When treating extensive third-degree burns, in which autologous skin that can be harvested for grafting is extremely limited, a cultured epidermal grafting technique or a thin meshed autografting technique is employed. However, survival of cultured epidermis grafted on a third-degree wound surface is poor, and the thin meshed autografting technique tends to easily cause disfiguring scarring or scar contracture. It is thought that such poorness of survival and formation of disfiguring scarring and contracture are mainly due to the lack of dermal components on the wound surface. It is therefore important to construct a new dermal structure by adding the dermal components to the wound surface at the same time as carrying out the thin meshed autografting technique or the cultured epidermal grafting technique (e.g., Matsuda, T. and Takami, Y., ‘Classification and Problems of Dermal Substitutes’, Burns, 1994, Vol. 20, No. 5, p. 221-230).

Various types of dermal substitute have been proposed and tried for this purpose. They can be broadly divided into artificial collagen matrixes employing xenogeneic collagen and acellular dermal matrixes formed by processing allogeneic skin. Some of the artificial collagen matrixes are available as commercial products in Japan and in the USA (Pelnac (trademark): Kowa Shinyaku Co., Ltd., Terudermis (trademark): Terumo Corporation, and Integra (trademark): Integra Lifescience, USA), but they are not suitable for grafting simultaneously with skin graft overlaying since they require a long time for vascularization after grafting.

Since the acellular dermal matrixes use cryopreserved human allogeneic skin as a starting material, they have a more physiological matrix structure than the artificial collagen matrixes, and it is known that the acellular dermal matrixes can be grafted simultaneously with a skin graft. Such an allogeneic acellular dermal matrix has been commercialized in the USA (AlloDerm (trademark): LifeCell Corporation, USA), but its preparation method is not necessarily ideal, and its clinical reliability is not satisfactory.

Under such circumstances the development of a more highly reliable allogeneic acellular dermal matrix that can be grafted simultaneously with an autologous skin graft has been awaited.

When preparing the above kind of allogeneic acellular dermal matrix, the decellularization method is extremely important, and as this method the following have been reported. Method 1: a method in which Dispase, which is a protease, and Triton X-100, which is a detergent, are employed in turn and, more specifically, a method in which, when split-thickness skin is separated into epidermis and dermis, a treatment with Dispase is carried out, and when the separated dermis is subsequently decellularized, a treatment with Triton X-100 alone is carried out (Takami, Y., Matsuda, T., Yoshitake, M., Hanumadas, M., and Walter, R. J., ‘Dispase/Detergent Treated Dermal Matrix as a Dermal Substitute.’, Burns, 1996, Vol. 22, No. 3, p. 182-190). Method 2: a method involving treatment with a 1 M sodium chloride solution and an SDS solution (Livesey, S. A., Henderson, D. N., Hollyoak, M. A., Atkinson, Y. H., and Nag, A., ‘Transplanted Acellular Allograft Dermal Matrix.’, Transplantation, 1995, Vol. 60, No. 1, p. 1-9). Method 3: a method in which freezing and thawing are repeated (Grillo, H. C. and Mckhann, C. F., ‘The acceptance and evolution of dermal homografts freed of viable cells’, Transplantation, 1964, Vol. 2, No. 1, p. 48-59). Method 4: a method involving simply treatment with trypsin (Oliver, R. F. and Grant, R. A., ‘Reconstruction of full-thickness loss skin wounds using skin collagen allografts’, Brit. J. Plastic Surgery, 1979, Vol. 32, No. 2, p. 87-90).

However, none of the above-mentioned conventional decellularization methods can be said to be successful in obtaining a highly reliable acellular dermal matrix suitable for simultaneous autografting of skin for reasons such as, for example, the degree of removal of cells being incomplete, or the dermal collagen structure being excessively denatured due to a long period of time being taken for treatment.

Furthermore, in the case where simultaneous grafting of a dermal substitute such as an acellular dermal matrix and an autologous skin graft overlay is carried out, there is a possibility that excessive regeneration of epithelium might be caused at the border between the autologous skin graft overlay and the dermal substitute (border epithelialization), thereby badly affecting wound healing, and it has been pointed out that this excessive and unwanted regeneration of epithelium might be promoted by biological components such as cells present on the dermal substitute (e.g., Yao, M., Takami, Y., and Ogo, K., ‘Effect of cultured dermal substitute composed of collagen sponge seeded with fibroblasts in simultaneous skin graft overlay.’, J. Kyorin Med. Soc., 2001, Vol. 32, No. 1, p. 59-69). Moreover, in accordance with various preliminary investigations by the present inventors, it has been found that, in the case where an acellular dermal matrix is used as the dermal substitute, when basement membrane components present at the dermis/epidermis border are more preserved, the epithelialization at the border tends to be more strongly promoted. Therefore, when simultaneous grafting with an autologous skin graft overlay is taken into consideration, there has been a desire for a method for decellularizing a dermal matrix that can attenuate residual basement membrane components.

Furthermore, not only the human allogeneic acellular dermal matrix but also an acellular dermal matrix originating from skin harvested from a xenogeneic mammal have a more physiological matrix structure than the artificial collagen matrix, and they are expected to be capable of being grafted simultaneously with a skin graft.

### BRIEF SUMMARY OF THE INVENTION

An object of the present invention is therefore to provide a highly reliable acellular dermal matrix and, in particular, an optimum allogeneic or xenogeneic acellular dermal matrix for simultaneous grafting with an autologous skin graft overlay, and it is also an intention of the present invention to provide a method of further utilizing the acellular dermal matrix.

As a result of an intensive investigation by the present inventors in order to solve the above-mentioned problems, it has been found that, by treating separated dermis with a protease and a surfactant simultaneously, it is possible to decellularize the dermis substantially completely in a short period of time without greatly denaturing the intrinsic collagen structure of the dermis while attenuating the residual basement membrane components. A first aspect of the present invention is therefore a method for decellularizing separated skin, the method comprising treating simultaneously with a protease and a surfactant (detergent).

As a preferred protease trypsin is used, and as the surfactant a polyoxyethylene p-t-octylphenyl ether surfactant can be used. Second and third aspects of the present invention are therefore the above-mentioned method wherein the protease is trypsin, and the above-mentioned method wherein the surfactant is a polyoxyethylene p-t-octylphenyl ether surfactant.

The above-mentioned decellularization method can be applied to the production of an acellular dermal matrix without any modification, and it has been confirmed that, in an acellular dermal matrix obtained according thereto, substantially complete decellularization is achieved, a normal three-dimensional intradermal collagen structure is adequately retained, and residual basement membrane components, which accelerate unwanted epithelialization, are reduced. That is, use of the decellularization method of the present invention enables the production of a more reliable acellular dermal matrix and, in particular, an allogeneic acellular dermal matrix that can be grafted simultaneously with an autologous skin graft overlay. Fourth to eleventh aspects of the present invention are therefore an acellular dermal matrix decellularized by treating simultaneously with a protease and a surfactant, the above-mentioned acellular dermal matrix wherein the protease is trypsin, the above-mentioned acellular dermal matrix wherein the surfactant is a polyoxyethylene p-t-octylphenyl ether surfactant, the above-mentioned acellular dermal matrix wherein human allogeneic skin is used as a starting material, the above-mentioned acellular dermal matrix wherein porcine skin is used as a starting material, a method for producing an acellular dermal matrix, the method comprising a decellularizing step of treating separated skin simultaneously with a protease and a surfactant, the above-mentioned method wherein the protease is trypsin, the above-mentioned method wherein the surfactant is a polyoxyethylene p-t-octylphenyl ether surfactant, the above-mentioned method wherein human allogeneic skin is used as a starting material, and the above-mentioned method wherein porcine skin is used as a starting material.

The above-mentioned acellular dermal matrix can desirably be used in the preparation of composite cultured skin (or Tissue Engineered Skin), in which epidermal cells and fibroblast cells are cultured using the matrix as a substrate. The present invention therefore also include a composite cultured skin employing as a substrate the above-mentioned acellular dermal matrix.

In accordance with each of the above-mentioned constitutions, the present invention is able to provide a highly reliable acellular dermal matrix, in particular an allogeneic acellular dermal matrix having an improved survival rate for a simultaneously grafted autologous skin graft and, moreover, an allogeneic acellular dermal matrix that can reduce scarring of a simultaneously overlaid meshed autologous skin graft and, in addition, provides a composite cultured skin employing the acellular dermal matrix as a substrate.

The present disclosure relates to subject matter contained in Japanese Patent Application No. 2002-276048, filed on Sep. 20, 2002, the disclosure of which is expressly incorporated herein by reference in its entirety.

### BRIEF DESCRIPTION OF THE DRAWINGS

FIGS. 1A and 1B are photographic diagrams showing the histology of the acellular dermal matrix of the present invention (HE staining, magnification 100 times). FIG. 1A: Allogeneic human skin (before treatment), FIG. 1B: after the decellularization treatment of the present invention (allogeneic acellular dermal matrix).

FIG. 2 is a photographic diagram showing an electron microscope image (magnification 6000 times) of the acellular dermal matrix of the present invention.

FIG. 3 is a photographic diagram showing the histology of the acellular dermal matrix of the present invention (PAS staining, magnification 100 times).

FIG. 4 is a photographic diagram showing the histology of the acellular dermal matrix of the present invention (anti-laminin immunohistochemistry, magnification 100 times).

FIG. 5 is a photographic diagram showing the histology of the acellular dermal matrix of the present invention (anti-type IV collagen immunohistochemistry, magnification 100 times).

FIG. 6 is a photographic diagram showing the histology of an acellular dermal matrix prepared by the freeze-thaw method (Method 3) (anti-type IV collagen immunohistochemistry, magnification 100 times).

FIGS. 7A and 7B are schematic diagrams of simultaneous grafting (rat model) of acellular dermal matrix and split-thickness skin graft. FIG. 7A: A schematic diagram of a grafting method on a rat dorsal full-thickness skin defect, FIG. 7B: a cross-sectional schematic diagram of the grafting method. In FIG. 7A, 1 denotes a SD rat, 2 denotes a site where only split-thickness skin grafting was carried out, and 3 denotes a site where overlay grafting of an acellular dermal matrix and a split-thickness skin graft was carried out. In FIG. 7B, B-1 is a cross-sectional schematic diagram of the site where only split-thickness skin grafting was carried out, and B-2 is a cross-sectional schematic diagram of the site where overlay grafting of the acellular dermal matrix and the split-thickness skin graft was carried out. In FIG. 7B, 4 denotes the split-thickness skin graft, 5 denotes the full-thickness skin defect, and 6 denotes the acellular dermal matrix.

FIGS. 8A and 8B are photographic diagrams showing images of simultaneous grafting (rat model) of the acellular dermal matrix of the present invention and a split-thickness skin graft overlay. FIG. 8A: Observation immediately after grafting, FIG. 8B: observation 1 week after grafting. In the figures, 2 denotes a site where only split-thickness skin grafting was carried out, and 3 denotes a site where overlay grafting of an acellular dermal matrix and a split-thickness skin graft was carried out.

FIG. 9 is a photographic diagram showing an image of simultaneous grafting (rat model) of the acellular dermal matrix of the present invention and the split-thickness skin graft overlay. Observation 8 weeks after grafting. In the figure, 2 denotes a site where only split-thickness skin grafting was carried out, and 3 denotes a site where overlay grafting of an acellular dermal matrix and a split-thickness skin graft was carried out.

FIGS. 10A and 10B are photographic diagrams showing images of simultaneous graft overlaying (rat model) of the acellular dermal matrix of the present invention and a split-thickness skin graft. Observation 1 week after grafting (HE staining, magnification 40 times). FIG. 10A: Split-thickness skin grafting only, FIG. 10B: overlay grafting of the acellular dermal matrix of the present invention and the split-thickness skin graft. In the figures, 7 denotes a grafted split-thickness skin section, and 8 denotes an acellular dermal matrix section.

FIGS. 11A and 11B are photographic diagrams showing images of simultaneous grafting (rat model) of the acellular dermal matrix of the present invention and the split-thickness skin graft. Observation 2 weeks after grafting (HE staining, magnification 100 times). FIG. 11A: Split-thickness skin grafting only, FIG. 11B: overlay grafting of the acellular dermal matrix of the present invention and the split-thickness skin graft. In the figures, 7 denotes a grafted split-thickness skin section, and 8 denotes an acellular dermal matrix section.

FIGS. 12A and 12B are photographic diagrams showing images of simultaneous graft overlaying (rat model) of the acellular dermal matrix of the present invention and the split-thickness skin graft. Observation 4 weeks after grafting (HE staining, magnification 100 times). FIG. 12A: Split-thickness skin grafting only, FIG. 12B: overlay grafting of the acellular dermal matrix of the present invention and the split-thickness skin graft. In the figures, 7 denotes a grafted split-thickness skin section, and 8 denotes an acellular dermal matrix section.

FIGS. 13A and 13B are photographic diagrams showing images of simultaneous grafting (rat model) of the acellular dermal matrix of the present invention and the split-thickness skin graft. Observation 8 weeks after grafting (HE staining, magnification 100 times). FIG. 13A: Split-thickness skin grafting only, FIG. 13B: overlay grafting of the acellular dermal matrix of the present invention and the split-thickness skin graft. In the figures, 7 denotes a grafted split-thickness skin section, and 8 denotes an acellular dermal matrix section.

FIG. 14 is a photographic diagram showing an image of simultaneous grafting of the allogeneic acellular dermal matrix of the present invention and a meshed split-thickness autologous skin graft. Clinical application case, 38-year-old female, thigh burn injury. Observation immediately before grafting.

FIG. 15 is a photographic diagram showing an image of simultaneous grafting of the allogeneic acellular dermal matrix of the present invention and the meshed split-thickness autologous skin graft overlay. Clinical application case, 38-year-old female, thigh burn injury. Observation immediately after grafting. In FIG. 15, 9 denotes an area where there was only the meshed split-thickness autologous skin graft, 10 denotes an area where overlay grafting of the acellular dermal matrix and the meshed split-thickness autologous skin graft was carried out, and 11 denotes the acellular dermal matrix.

FIG. 16 is a photographic diagram showing an image of simultaneous grafting of the allogeneic acellular dermal matrix of the present invention and the meshed split-thickness autologous skin graft. Clinical application case, 38-year-old female, thigh burn injury. Observation 7 days after grafting. In FIG. 16, 9 denotes the area where there was only the meshed split-thickness autologous skin graft, and 10 denotes the area where overlay grafting of the acellular dermal matrix and the meshed split-thickness autologous skin graft was carried out.

FIG. 17 is a photographic diagram showing an image of simultaneous grafting of the allogeneic acellular dermal matrix of the present invention and the meshed split-thickness autologous skin graft overlay. Clinical application case, 38-year-old female, thigh burn injury. Observation 14 days after grafting. In FIG. 17, 9 denotes the area where there was only the meshed split-thickness autologous skin graft, and 10 denotes the area where overlay grafting of the acellular dermal matrix and the meshed split-thickness autologous skin graft was carried out.

FIGS. 18A and 18B are photographic diagrams showing images of simultaneous grafting of the allogeneic acellular dermal matrix of the present invention and the meshed split-thickness autologous skin graft overlay. Clinical application case, 38-year-old female, thigh burn injury. Observation 21 days after grafting. In the figures, 9 denotes the area where there was only the meshed split-thickness autologous skin graft, and 10 denotes the area where overlay grafting of the acellular dermal matrix and the meshed split-thickness autologous skin graft was carried out. FIG. 18B is enlargement of FIG. 18A.

FIG. 19 is a photographic diagram showing an image of simultaneous grafting of the allogeneic acellular dermal matrix of the present invention and a meshed split-thickness autologous skin graft overlay. Clinical application case, histological observation 14 days after grafting (HE staining, magnification 100 times). In FIG. 19, 11 denotes the overlay-grafted split-thickness autologous skin graft, and 12 denotes the acellular dermal matrix.

FIG. 20 is a photographic diagram showing a histological observation (HE staining, magnification 200 times) 20 weeks after subcutaneous grafting of the acellular dermal matrix (rat model) of the present invention. In FIG. 20, 12 denotes the acellular dermal matrix.

FIGS. 21A, 21B and 21C are photographic diagrams showing histological observations of a composite cultured skin on the 14th day using the allogeneic acellular dermal matrix of the present invention as a substrate. HE stained image, FIG. 21A is magnified 100 times, and FIG. 21B and 21C are 200 times (magnified figure). In the figures, 13 denotes an epidermal cell layer, 14 denotes the acellular dermal matrix, and 15 denotes fibroblast cells.

### BEST MODE FOR CARRYING OUT THE INVENTION

The decellularization method of the present invention is applied to dermis that is obtained by using skin harvested from an allogeneic mammal, including man, and separating the skin into epidermis and dermis. The skin harvested from an allogeneic mammal used in the present invention is skin harvested from an animal that is allogeneic to an animal that requires a treatment such as skin grafting as a burn treatment, etc., and whether it is autologous or not does not matter as long as it is derived from an allogeneic animal. It is also possible to use surplus skin not needed after surgery or after an allogeneic skin harvest, cadaver skin, etc. and, furthermore, skin cryopreserved in a skin bank, etc. It is desirable to use the skin as split-thickness skin having an average thickness of about 0.38 mm (average thickness of about 0.015 inch).

Moreover, the decellularization method of the present invention is applied to dermis obtained by harvesting skin from a xenogeneic mammal and separating the skin into epidermis and dermis. Examples of the xenogeneic mammal used in the present invention include pig, cattle, monkey, rabbit, rat, mouse, goat, sheep, and horse, and in the present invention pig is preferably used. The skin harvested from these xenogeneic mammals is desirably used as a split-thickness skin having an average thickness of about 0.38 mm (average thickness of about 0.015 inch).

Separation of harvested skin into epidermis and dermis can be carried out by any method known to a person skilled in the art for this purpose, and it is preferably carried out, for example, by immersing surplus skin (split-thickness skin: average thickness 0.015 inch) not needed during surgery or after an allogeneic skin harvest, in a mixed solution containing 0.25 wt % trypsin and 1 mM EDTA and incubating at 37° C. for 3 hours. This treatment enables the epidermis and the dermis to be easily separated.

The dermis thus obtained is subsequently decellularized by the method of the present invention. That is, this dermis is treated with a mixed solution containing effective concentrations of a protease and a surfactant.

As the protease, any type known to a person skilled in the art in the field of dermal decellularization may be used without any problems. Two or more types of proteases can also be used in combination; examples thereof include trypsin and Dispase and, in particular, trypsin can accomplish excellent decellularization. With regard to the surfactant (detergent), any type known to a person skilled in the art in this field may be used without any problems; two or more types of surfactants (detergents) can be used as a mixture in some cases, and the surfactant (detergent) is preferably selected from those having an excellent membrane-solubilizing ability and exhibiting minimum denaturation/deactivation of a protein. Polyoxyethylene p-t-octylphenyl ether surfactants, represented by Triton X-100 (product name), are used as desirable surfactants (detergents).

The mixed solution for treating dermis referred to above is a solution that contains at least the active protease and the surfactant of the present invention, and can optionally contain another additional component such as, for example, EDTA, a buffer salt, a vitamin, a preservative, or an antibiotic. The concentration of the protease of the present invention in this mixed solution is 0.01 to 2.0 wt %, preferably 0.05 to 0.5 wt %, and particularly preferably 0.1 to 0.25 wt %, and the concentration of the surfactant of the present invention is 0.05 to 1.0 wt %, preferably 0.1 to 0.5 wt %, and particularly preferably 0.2 to 0.3 wt %.

Treatment of the dermis with the mixed solution includes immersion of the dermis in this mixed solution, and preferably includes immersion in this mixed solution and shaking in this mixed solution. The temperature at which the immersion and shaking may be a temperature at which there is substantially no denaturation or deactivation of the protease of the present invention and the treated dermis; they are generally carried out at 20° C. to 37° C. but the temperature is not limited thereto. Furthermore, under conditions in which the protease and the treated dermis are not substantially denatured/deactivated, in order to promote decellularization, sonication may additionally be carried out. About 3 to 4 hours of treatment is sufficient, but the treatment time can be reduced or may be set at a slightly longer time (e.g., about 5 hours) while taking into consideration the state of the decellularization.

In a preferred embodiment of the decellularization method, a dermis portion obtained by separation from epidermis is immersed in a mixed solution containing 0.125 wt % trypsin, 1 mM EDTA, and 0.25 wt % Triton X-100 and shaken at 37° C. for 3 to 4 hours. This treatment removes substantially all cellular components (including cells of cutaneous appendages, vascular cells, fibroblast cells, and nervous system cells) within the dermis, and the dermis thus obtained is composed only of a dermal matrix containing collagen as the main constituent.

Furthermore, the decellularization method of the present invention can include, in addition to the above-mentioned steps, a step of sterilizing the separated skin or the decellularized separated skin by immersing it in an approximately 0.1% to 10% aqueous solution of sodium azide for several minutes to several days. Moreover, a step of sterilizing the separated skin or the decellularized separated skin by irradiating it with gamma rays or an electron beam can be included in any stage of the decellularization method of the present invention. Alternatively, these sterilization steps can be applied to the starting skin. The decellularization method of the present invention can also include any other step.

The dermis (matrix) thus decellularized is washed well, preferably with a biocompatible solution such as a phosphate buffered saline. The dermal matrix thus washed can be used as the acellular dermal matrix of the present invention without further treatment, or it can be frozen for storing before use.

Furthermore, it is more preferable to subject a part of the acellular dermal matrix obtained above to bacterial and fungal culture, thereby confirming that there is no growth of bacteria or fungi. It is yet more preferable to carry out a pathological test involving hematoxylin eosin staining, thereby confirming that there is substantially no abnormality in the dermal collagen structure and the dermis is substantially completely acellular.

The above-mentioned decellularization method/acellular dermal matrix production method is an excellent method that, compared with the conventional methods, can decellularize more reliably in a shorter time while retaining the normal dermal matrix structure.

The acellular dermal matrix produced above is substantially acellular and retains a normal three-dimensional intradermal collagen structure. Furthermore, there is remarkably little damage to the intradermal collagen structure, whereas sections of the epidermis/dermis border corresponding to the basement membrane are not densely stained by PAS staining, suggesting that expression of laminin and type IV collagen, which are basement membrane components, is attenuated.

That is, this acellular dermal matrix provides a dermal substitute that can be used clinically as a human allogeneic acellular dermal matrix or a xenogeneic acellular dermal matrix. In clinical application, for example, the above-mentioned simultaneous skin graft overlaying, in which the dermal component is added at the same time as a thin autologous skin graft, can be cited as a standard application method, and the acellular dermal matrix can also be used instead of a xenogeneic collagen implant as a subcutaneous tissue expander for correcting depressed scarring, as a substitute for artificial dura mater for dura mater defect repair, as a substitute for an artificial mesh material for abdominal wall reinforcement and, furthermore, as a graftable composite cultured skin (Tissue engineered skin) substrate for replacing the xenogeneic collagen matrix.

In particular, with regard to the composite cultured skin, development of composite cultured skin (or Tissue engineered skin) formed by culturing epidermal cells and fibroblast cells using a dermis-like matrix as a substrate has been carried out, and the cases reported so far mainly employ a bovine- or porcine-derived collagen gel or collagen sponge as a matrix. Since these matrixes do not have a physiological dermal collagen structure, it is known that the survival and stability thereof are clinically poor. On the other hand, the human allogeneic acellular dermal matrix of the present invention can be used as a good substrate for normal human skin derived epidermal cells and fibroblast cells and, furthermore, layering of an epidermal portion is obtained in a few days of culturing. It has also been confirmed that the composite cultured skin prepared in this way exhibits a dermal matrix structure that is very similar to normal human skin. Moreover, the xenogeneic acellular dermal matrix of the present invention can also be used as a good substrate for normal human skin derived epidermal cells and fibroblast cells and, furthermore, layering of an epidermal portion is obtained in a few days of culturing.

The method for producing the acellular dermal matrix of the present invention is an excellent method that, compared with the conventional methods, can decellularize reliably in a short time while retaining a normal dermal matrix structure. A human allogeneic or xenogeneic acellular dermal matrix prepared according to the method of the present invention is a clinically usable dermal substitute. In clinical application, the above-mentioned simultaneous skin grafting, in which a dermal component is added at the same time as a thin autologous skin graft, can be cited as a standard application method, and the acellular dermal matrix can also be used instead of a xenogeneic collagen implant as a subcutaneous tissue expander for correcting depressed scarring, as a substitute for artificial dura mater for dura mater defect repair, as a substitute for an artificial mesh material for abdominal wall reinforcement and, furthermore, as a graftable composite cultured skin (Tissue engineered skin) substrate for replacing the xenogeneic collagen matrix, etc.

The present invention is explained in further detail by Examples, but the present invention is of course not limited only to these Examples.

### EXAMPLES

### Example 1

### Preparation of Acellular Dermal Matrix

Treatment 1: Separation into Epidermis and Dermis

Surplus skin (split-thickness skin: average thickness of about 0.38 mm: 0.015 inch) not needed during surgery or after harvesting allogeneic skin was immersed in a mixed solution containing 0.25 wt % trypsin and 1 mM EDTA and incubated at 37° C. for 3 hours. This treatment easily separated dermis from epidermis.

Treatment 2: Removal of Cellular Components Within Dermis

A dermis portion obtained in Treatment 1 was immersed in a mixed solution containing 0.125 wt % trypsin, 1 mM EDTA, and 0.25 wt % Triton X-100 (product name) and shaken at 37° C. for 3 to 4 hours. This treatment removed all of the cellular components (including cells of cutaneous appendages, vascular cells, fibroblast cells, and nervous system cells) within the dermis, and the dermis was composed only of a dermal matrix containing collagen as the main constituent.

Treatment 3: Washing, Testing, and Storage of Acellular Dermal Matrix

The acellular dermal matrix obtained in Treatment 2 was washed well with a phosphate buffered saline and then frozen for storage. At the same time as this, it was confirmed that there was no growth of bacteria or fungi by subjecting a part of the dermal matrix to bacterial and fungal culture. Furthermore, it was confirmed by a pathological test involving hematoxylin eosin staining that there was no abnormality in the dermal collagen structure and that the dermis was completely acellular.

### Example 2

### Histological Properties of Acellular Dermal Matrix

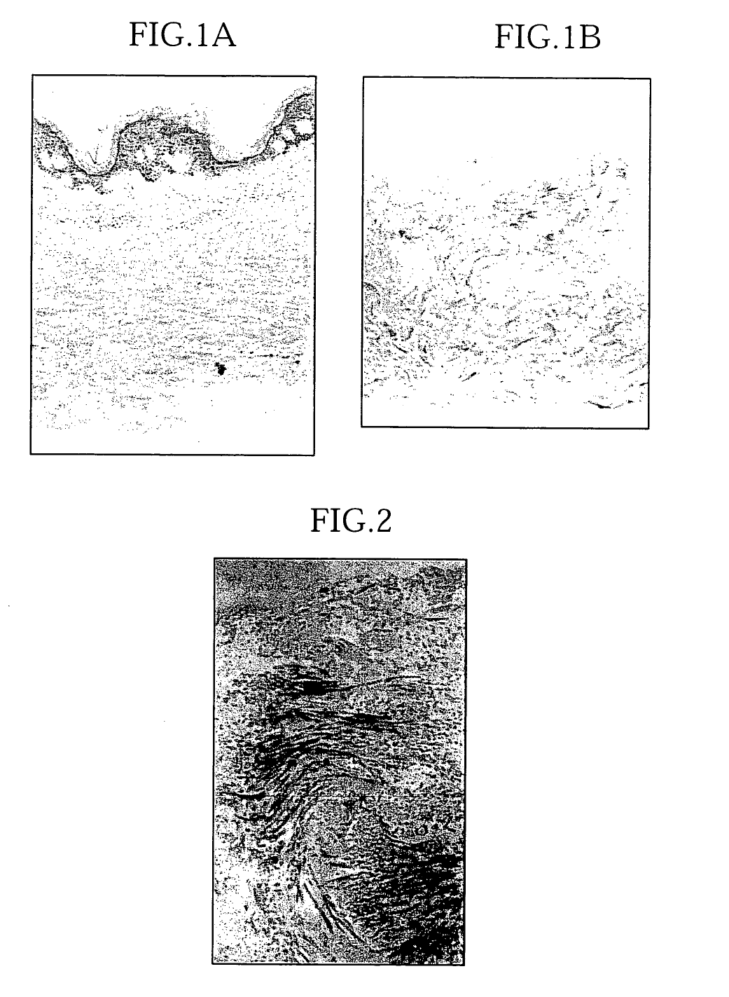

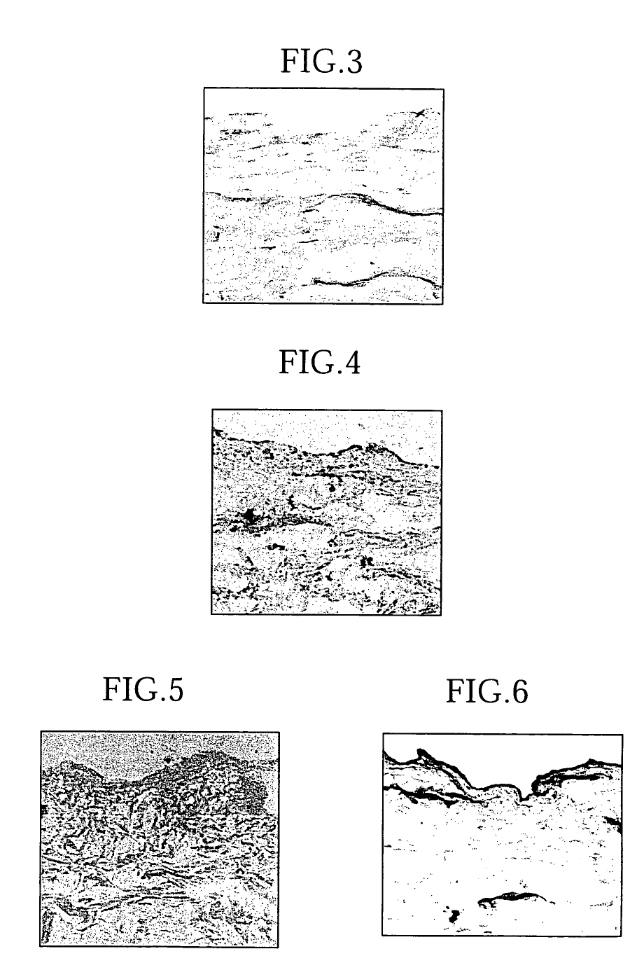

The acellular dermal matrix prepared as above was completely acellular and retained a normal three-dimensional intradermal collagen structure (FIGS. 1a and 1b). Microscopic observation showed that there was very little damage to the intradermal collagen structure (FIG. 2). On the other hand, a section of the epidermis/dermis border corresponding to the basement membrane was not stained by PAS staining (FIG. 3), and expression of laminin and type IV collagen, which are components of the basement membrane, was attenuated (FIGS. 4, 5, and 6).

### Example 3

### Comparative Study of the Acellular Dermal Matrix of the Present Invention and Acellular Dermal Matrix from Conventional Methods

In order to examine the characteristics and advantages of the production method for the acellular dermal matrix of the present invention, comparison thereof with decellularization methods reported in the art was carried out. As the conventional methods reported so far, there are the following methods.
Method 1: a method in which Dispase, which is a protease, and Triton X-100, which is a detergent, are employed in turn and, that is, a method in which, when split-thickness skin is separated into epidermis and dermis, a treatment with Dispase is carried out, and when the separated dermis is subsequently decellularized, a treatment with Triton X-100 alone is carried out (Y. Takami et al, Burns, 1996, Vol. 22, No. 3, p. 182-190).
Method 2: a method involving treatment with a 1 M sodium chloride solution and an SDS solution (S. A. Livesey et al., Transplantation, 1995, Vol. 60, No. 1, p. 1-9).
Method 3: a method in which freezing and thawing are repeated (H. C. Grillo et al., Transplantation, 1964, Vol. 2, No. 1, p. 48-59).
Method 4: a method involving simply treatment with trypsin (R. F. Oliver et al., Brit. J. Plastic Surgery, 1979, Vol. 32, No. 2, p. 87-90).

The separated skin was treated by each of these methods, and comparison with the treatment method of the present invention (hereinafter called ‘Method 5’) was made.

3-1: Extent of Decellularization

The extent and ease of decellularization were studied. It was found that, in Methods 1, 2, 4, and 5 cells were completely removed. However, in Method 3 it was observed that a small number of cellular components remained.

3-2: Treatment Time

Reducing the treatment time required for decellularizing skin results in as little denaturing as possible of the dermal collagen structure. The treatment time to decellularization by each of various treatment methods was about 13 hours for Method 1, about 13 hours for Method 2, at least 48 hours for Method 3, and at least 7 days for Method 4, but in Method 5 (the present invention) all the steps were completed in about 8 hours. It is surmised from these results that the treatment method of the present invention is an excellent method for decellularizing skin in the shortest period of time.

3-3: Amount of Basement Membrane Components Remaining

The amount of basement membrane components of the dermis/epidermis border remaining was examined by immunohistochemistry employing the amount of type IV collagen remaining as an index. It was found that in Methods 1, 2, and 3 the expression of type IV collagen was strongly detected, but in Method 5 (the present invention) and Method 4 the expression of type IV collagen was attenuated, and it was confirmed that little of the basement membrane structure remained (FIGS. 5 and 6).

3-4: Survival Rate of Simultaneous Skin Graft Overlays

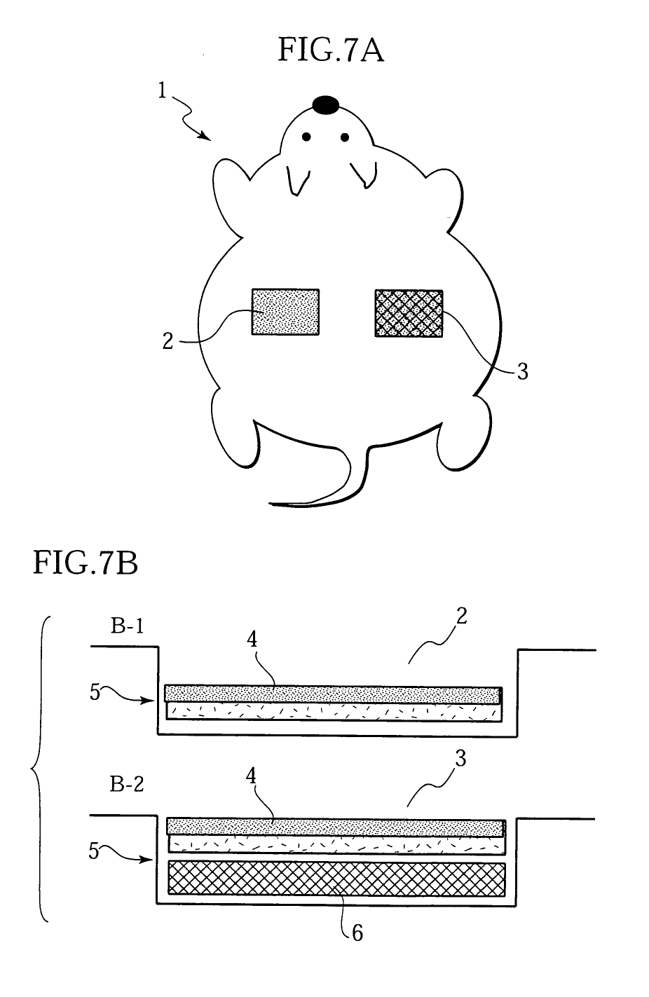

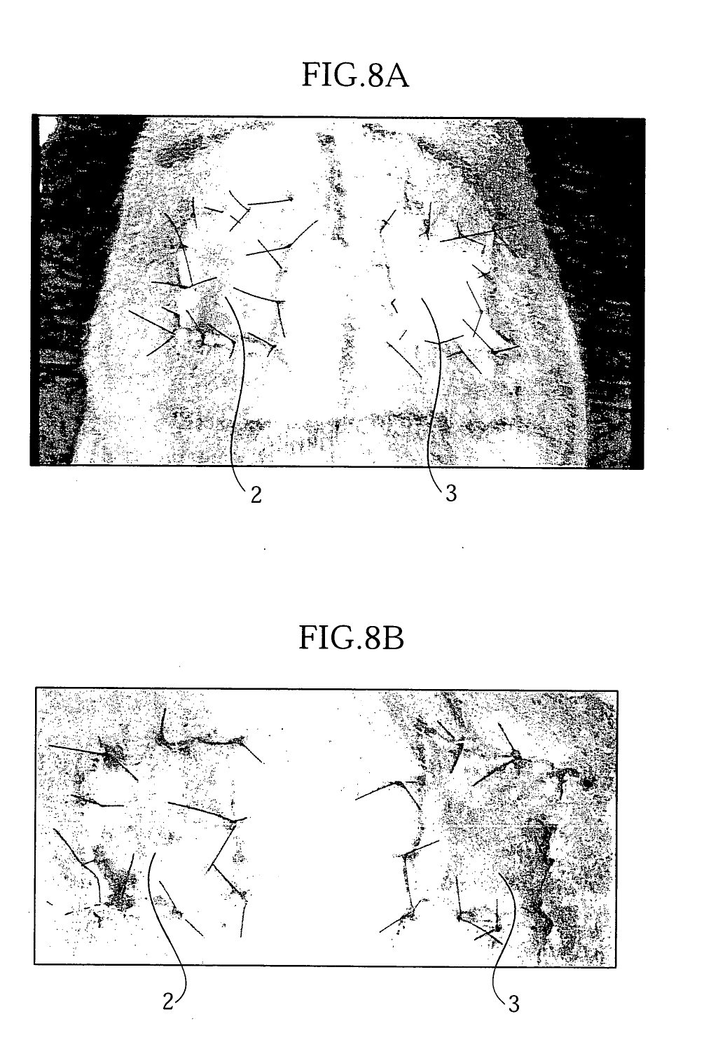

The simultaneous skin graft overlay and the acellular dermal matrix prepared by the treatment method of the present invention was carried out using rat models, and the survival rate of the skin graft was examined by the method of Takami et al. (Burns, 1996, Vol. 22, No. 3, p. 182-190) (n=20). Two full-thickness dorsal skin defects (2×2 cm square) were formed on Male SD rats. Grafted on one of the defects was a SD rat split-thickness skin (isograft) (split-thickness skin grafting), and overlay grafted on the other of the defects were firstly acellular dermal matrixes and simultaneously SD rat split-thickness skin graft overlays (isografts) (FIGS. 7A and 7B). It was found that the skin graft survival rate in the area where there was only the split-thickness skin graft was 92.0%, whereas the survival rate of skin grafted simultaneously on the acellular dermal matrix was an average of 86.8% (FIGS. 8A and 8B). There was no statistically significant difference between these two survival rates.

The survival rates of skin grafted simultaneously with dermal matrixes prepared by the above-mentioned various methods were as follows. Method 1: 64.3%, Method 3: 73.8%, and Method 4: 65.7%. With regard to Method 2, the data given in the literature was 73.2% (S. A. Livesey et al., Transplantation, 1995, Vol. 60, No. 1, p. 1-9). That is, the survival rates of the split-thickness skin graft overlaid simultaneously with the acellular dermal matrixes treated by the conventional methods were in all cases inferior to the survival rate obtained using the acellular dermal matrix prepared by the treatment method of the present invention. It is surmised that, as hereinbefore described, since the method of the present invention has a shorter treatment time than that of the other conventional methods, there is little denaturation of the dermal collagen structure, and since there are few basement membrane components in the cortical layer, there are few bad effects such as epithelialization at the border.

It is concluded from the above-mentioned examination that, compared with the conventional treatment methods, the method of the present invention is a method that can carry out decellularization simply, easily, and reliably in a short treatment time, and is an optimal method for preparing a dermal matrix for simultaneous grafting with an autologous skin graft.

### Example 4

### Characteristics of the Acellular Dermal Matrix of the Present Invention

4-1: Bacteriological Safety and Cytotoxicity

The human allogeneic acellular dermal matrix obtained by the method of the present invention did not show any microbiological growth of bacteria or fungi. It was found from incubating the acellular dermal matrix with skin-related cultured cells such as cultured human epidermal cells, fibroblast cells, and vascular endothelial cells, that no cytotoxicity was observed in any of the cells.

4-2: Graft Characteristics by Animal Experiment

(4-2-1) Simultaneous Grafting of Acellular Dermal Matrix and Split-Thickness Skin Graft in Full-Thickness Skin Defect: Survival Rate Thereof and Progress After Grafting

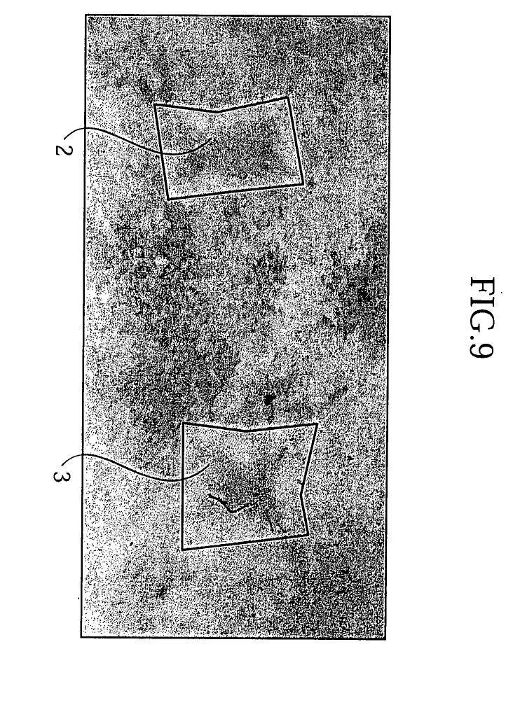

Two 2×2 cm full-thickness dorsal skin defects were formed on 8 week-old male SD rats (n=8), a 2×2 cm human acellular dermal matrix of the present invention was grafted on one of the wounds, and a 0.25 mm thick identically sized SD rat split-thickness skin graft was grafted thereover. As a control, the other wound was subjected only to SD rat split-thickness skin grafting (FIGS. 7A and 7B). Survival rates of the split-thickness skin grafts one week after grafting were examined by the method of Takami et al. (Burns, 1996, Vol. 22, No. 3, p. 182-190). It was found that the survival rate when carrying out simultaneous grafting on the acellular dermal matrix of the present invention was 86.8±10.3% (average value±standard deviation, the same applies below), and there was no statistically significant difference from the survival rate (91.9±4.7%) obtained by split-thickness skin grafting alone (FIGS. 8A and 8B). Although gradual contraction of the wound in the grafted part was observed until 4 weeks after the grafting, after that the wound became stable and presented a fully grown skin graft picture 8 weeks after the grafting (FIG. 9).

(4-2-2) Simultaneous Grafting of Acellular Dermal Matrix and Split-Thickness Skin Graft Overlay in Full-Thickness Skin Defect (Histology Thereof)

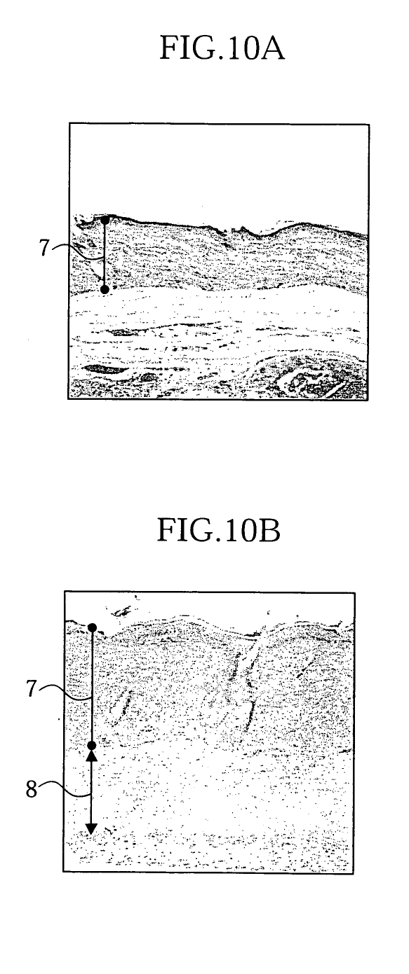

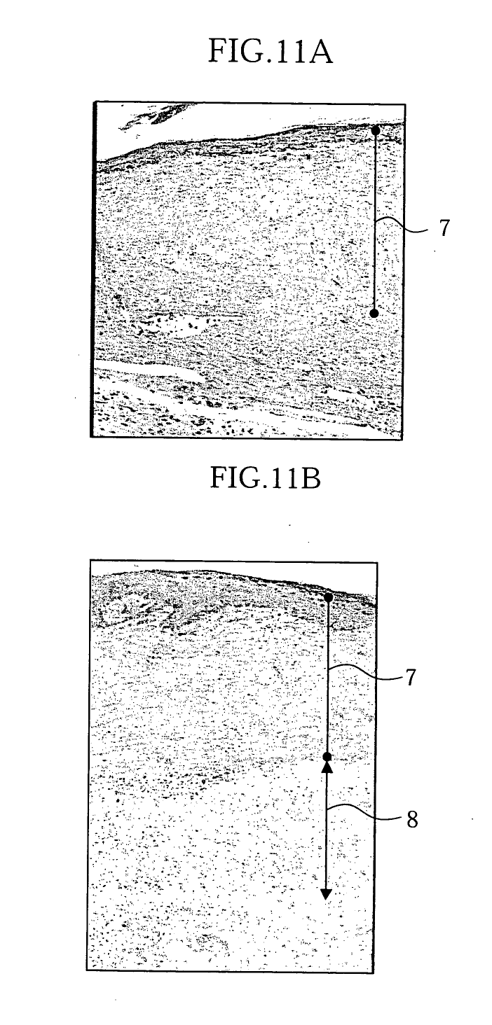

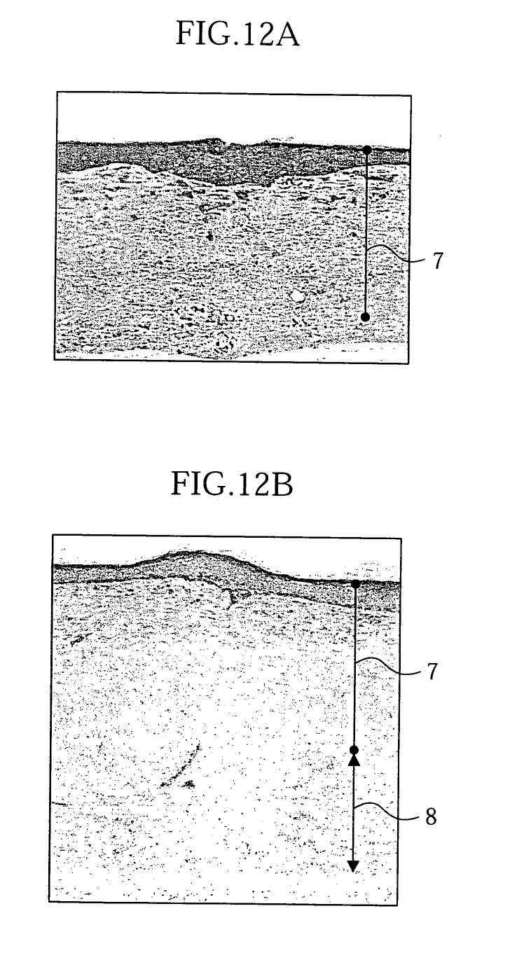

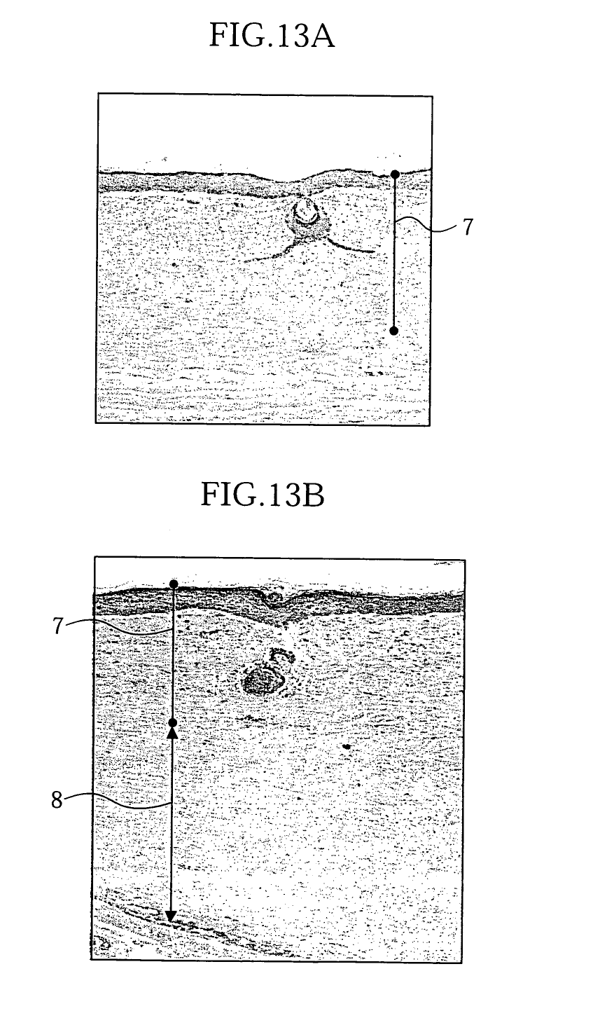

The grafted portion produced in 4-2-1 was biopsied over time, and a histological examination was carried out. At the one week stage after grafting, phlogocyte infiltration and vascularization from the underlying bed into the grafted acellular dermal matrix were observed. The dermal collagen matrix structure was retained well. There was no denaturing in the upper layer rat split-thickness skin, and it was observed histologically that the graft was surviving (FIGS. 10A and 10B). Two weeks after grafting vascularization within the acellular dermal matrix increased further, and adequate graft survival was shown. The border between the grafted acellular dermal matrix and its upper layer split-thickness rat skin became obscure, and it was observed that a new dermis-like structure in which the two were united was being formed (FIGS. 11A and 11B). Four weeks after grafting, this tendency intensified, and the inflammatory cell infiltration subsided (FIGS. 12A and 12B). Eight weeks after grafting, the new dermis-like structure was complete, and showed an image similar to a normal dermal structure (FIGS. 13A and 13B). During this observation period, no immunological rejection toward the graft was observed. Compared with the control, which employed grafting of split-thickness skin alone, there was no obvious difference in the graft survival process of the rat split-thickness skin grafted on the grafted acellular dermal matrix.

(4-2-3) Simultaneous Grafting of Acellular Dermal Matrix and Split-Thickness Skin Graft Overlay in Full-Thickness Skin Defect: Effect in Retaining New Dermis-Like Structure

The thickness of the dermis-like structure portion on the wound surface after grafting was measured using the histological sample prepared in 4-2-2. It was found that 4 weeks after grafting the thickness was 0.72±0.03 mm (average value±standard deviation, the same applies below) for the grafting of split-thickness skin alone and 0.90±0.10 mm for the simultaneous grafting of the acellular dermal matrix of the present invention and the split-thickness skin, and 8 weeks after grafting the thickness was 0.65±0.07 mm for the grafting of the split-thickness skin alone and 0.78±0.03 mm for the simultaneous grafting of the acellular dermal matrix and the split-thickness skin overlay. In both time periods, the simultaneous graft of the acellular dermal matrix and the split-thickness skin showed the formation of a statistically significantly thicker dermis-like structure (n=3 for each time period). These results confirm that grafting of the acellular dermal matrix of the present invention has the effect of retaining a new dermis-like structure for a long period of time.

(4-2-4) Simultaneous Grafting of Acellular Dermal Matrix and Split-Thickness Skin Graft Overlay in Full-Thickness Skin Defect (Effect in Suppressing Wound Contraction)

Wound areas of the skin grafts prepared in 4-2-1 were measured when grafting and 4 weeks after grafting, and the wound contraction was examined (n=5). The wound contraction was expressed as the size of the wound (graft) remaining 4 weeks after grafting and was obtained from the following equation. Percentage remaining=(Wound area 4 weeks after grafting/Wound area when grafting)×100. The percentages remaining were 34.1±6.0% (average value±standard deviation, the same applies below) for grafting of split-thickness skin alone, and 58.5±8.5% for simultaneous grafting of the acellular dermis matrix and the split-thickness skin, and it was observed that wound contraction was significantly suppressed in the acellular dermal matrix graft group.

### Example 5

### Clinical Application

5-1: Simultaneous Grafting of Acellular Dermal Matrix and Split-Thickness Skin Graft Overlay

In accordance with the guidelines of Kyorin University Ethics Committee, the acellular dermal matrix of the present invention was grafted on a part of the wound of each of three severe burn cases, where consent had been obtained, and it was simultaneously covered with autologous meshed split-thickness skin graft (Table 1).

TABLE 1
List of clinical application cases

| | | | | Split- | | |
|---|---|---|---|---|---|---|
| | | | | thickness | | |
| | Age/ | Wound | | skin | Graft | Mesh |
| Case | sex | surface | ADM* | graft | survival | scar |
| 1 | 38/F | Thigh | 7 × 5 cm | 0.20 mm | Complete | Improved |
| | | 3rd-degree | | | | |
| | | burn | | | | |
| 2 | 64/M | Dorsal | 3 × 3 cm | 0.25 mm | Complete | Improved |
| | | 3rd-degree | | | | |
| | | burn | | | | |
| 3 | 27/M | Thigh | 5 × 5 cm | 0.25 mm | Complete | Improved |
| | | 3rd-degree | | | | |
| | | burn | | | | |

*ADM: Allogeneic acellular dermal matrix

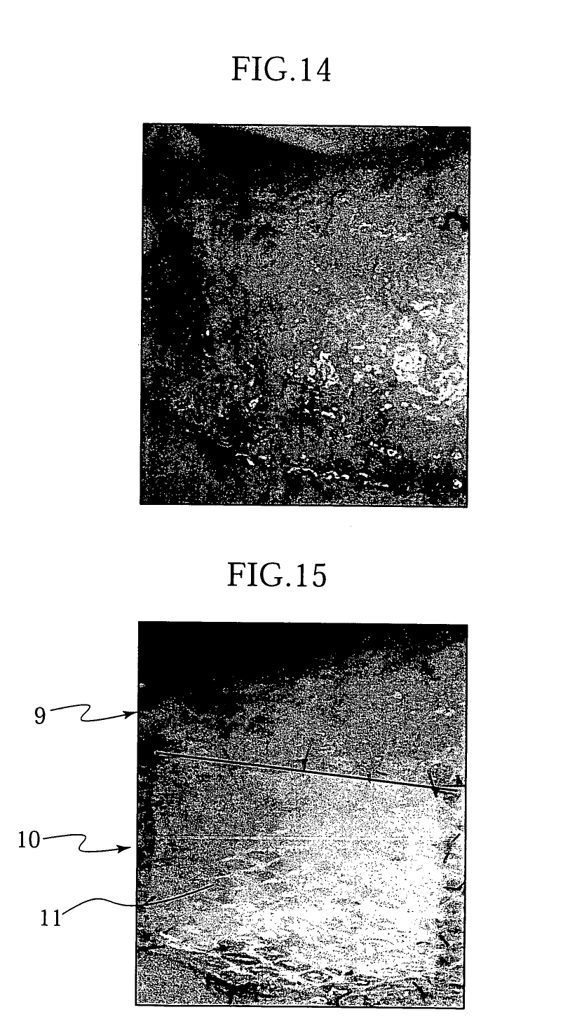

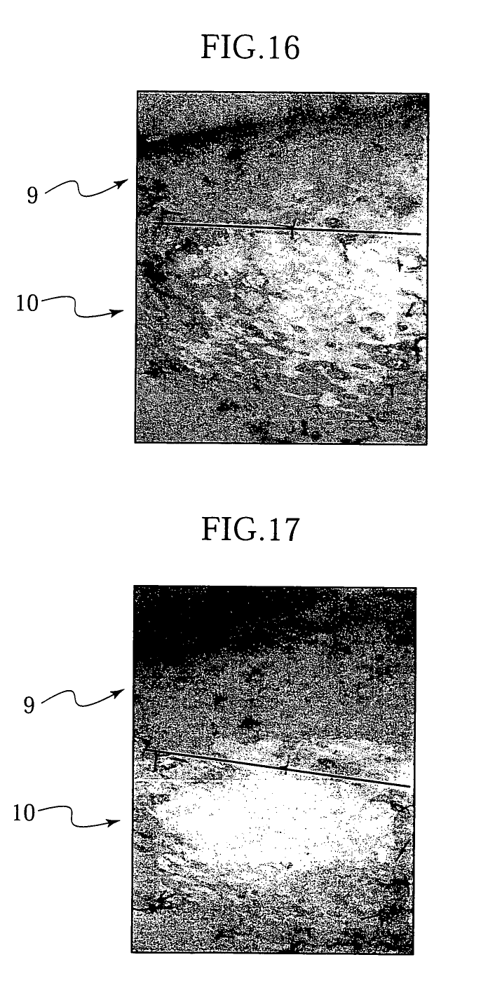

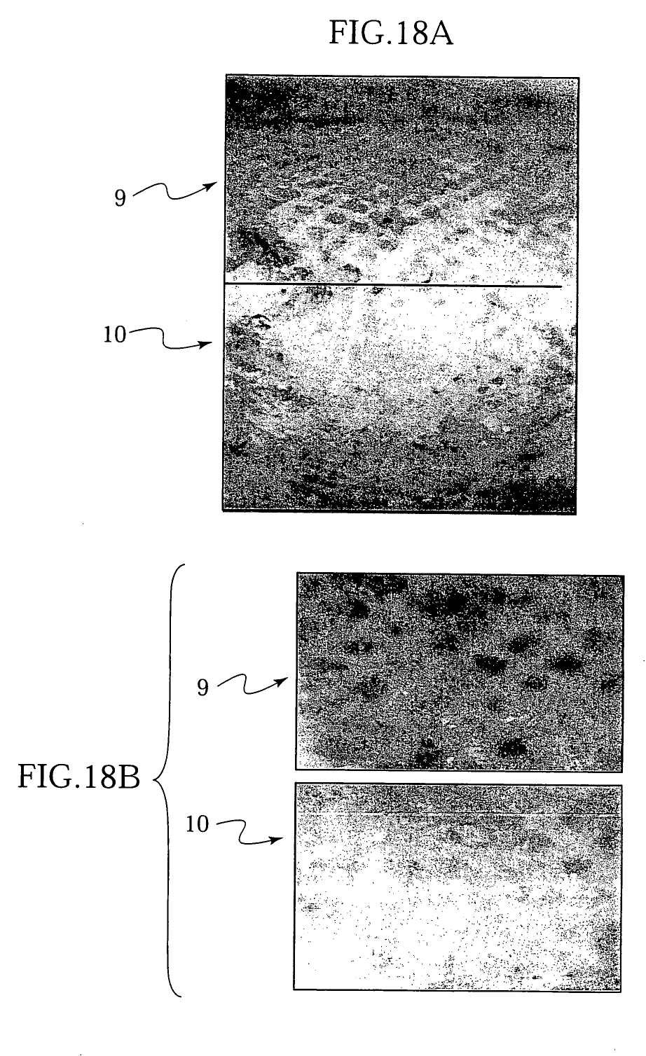

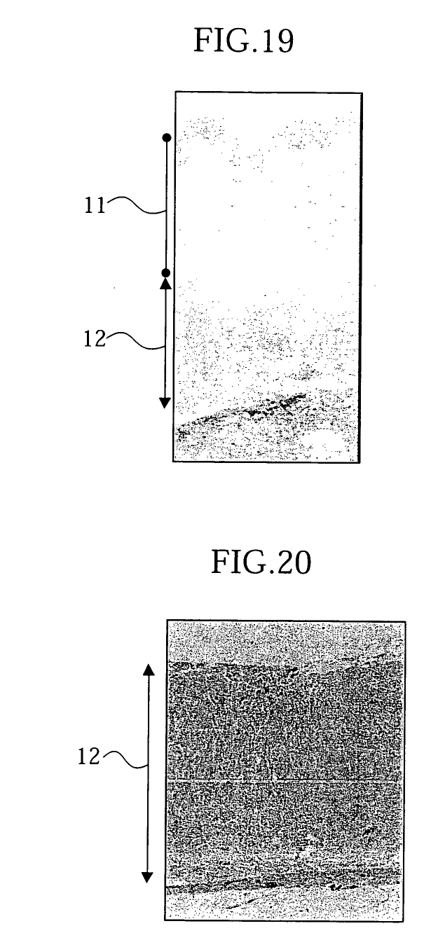

The grafted acellular dermal matrix and autologous skin graft survived in good condition in all cases, and it was shown that the acellular dermal matrix can function as a dermal template. Furthermore, there was a tendency for the meshed skin graft scarring to be further reduced (FIGS. 14, 15, 16, 17, 18A, and 18B). In biopsy histology two weeks after surgery vascularization was observed in both the grafted acellular dermal matrix and the split-thickness skin graft, suggesting that they were surviving completely (FIG. 19).

5-2: Subcutaneous Grafting of Acellular Dermal Matrix

In order to examine the grafting characteristics when the acellular dermal matrix of the present invention was grafted subcutaneously, subcutaneous dorsal grafts of 2×2 cm squares of the acellular dermal matrix were carried out on SD rats, and histological observations 4 weeks, 8 weeks, and 20 weeks after grafting were examined (n=3 for each time period). It was found that throughout the observation period, local inflammation was not detected visually, and the grafted acellular dermal matrix remained in the grafted part. The remaining collagen matrix structure and vascularization within the acellular dermal matrix were observed histologically (FIG. 20). It is surmised from the above findings that a subcutaneous graft of the acellular dermal matrix can be a grafting material for a subcutaneous connective tissue defect.

5-3: Preparation of Composite Cultured Skin Using Acellular Dermal Matrix as Substrate

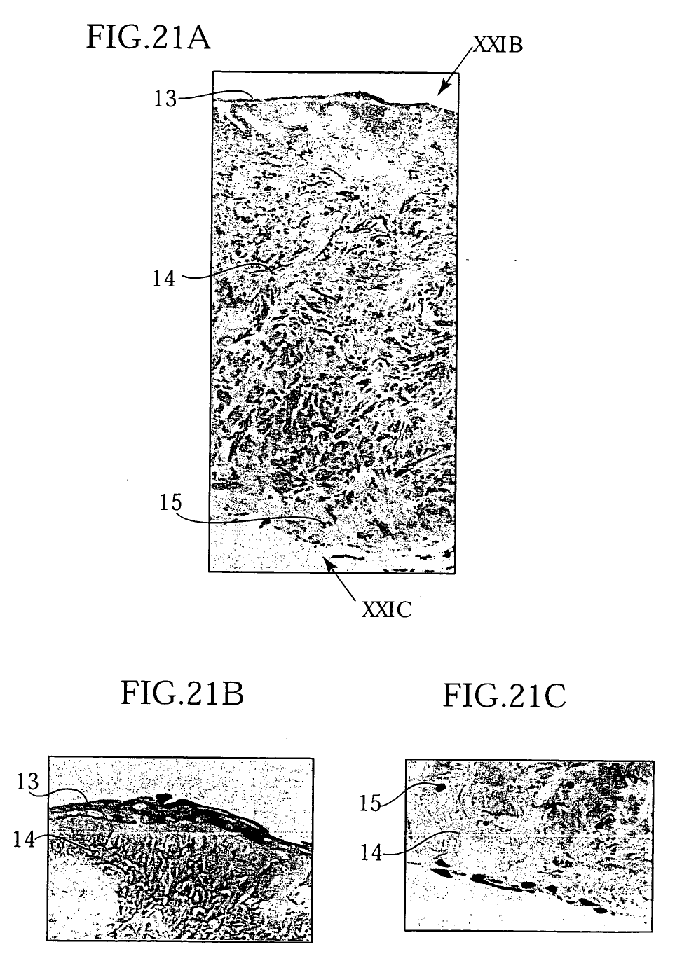

The development of a composite cultured skin (or Tissue engineered skin) in which epidermal cells and fibroblast cells are cultured using a dermis-like matrix as a substrate is advancing, and those reported so far mainly employ a bovine- or porcine-derived collagen gel or collagen sponge as a matrix. Since these matrixes do not have a physiological dermal collagen structure, it is known that the graft survival and stability thereof is clinically poor. An investigation has been carried out into whether or not the acellular dermal matrix of the present invention is effective as an excellent matrix that can replace the xenogeneic collagen matrix. It was found that normal human skin-derived epidermal cells and fibroblast cells were cultured well on the acellular dermal matrix. By further culturing for a few days, the epidermal portion became multi-layered. The composite cultured skin prepared in this way still had a thin epidermal cell layer, but it exhibited a dermal matrix structure that was very similar to normal human skin (FIGS. 21A, 21B and 21C).

### Example 6

### Preparation of Acellular Dermal Matrix

Treatment 1: Separation into Epidermis and Dermis

Porcine skin (split-thickness skin: average thickness of about 0.38 mm: 0.015 inch) is immersed in a mixed solution containing 0.25 wt % trypsin and 1 mM EDTA and incubated at 37° C. for 3 hours. This treatment easily separates dermis from epidermis.

Treatment 2: Removal of Cellular Components Within Dermis

A dermis portion obtained in Treatment 1 is immersed in a mixed solution containing 0.125 wt % trypsin, 1 mM EDTA, and 0.25 wt % Triton X-100 (product name) and shaken at 37° C. for 3 to 4 hours. This treatment removes all of the cellular components (including cells of cutaneous appendages, vascular cells, fibroblast cells, and nervous system cells) within the dermis, and the dermis is composed only of a dermal matrix containing collagen as the main constituent.

Treatment 3: Washing, Testing, and Storage of Acellular Dermal Matrix

The acellular dermal matrix obtained in Treatment 2 is washed well with a phosphate buffered saline and then frozen for storage. At the same time as this, it is confirmed that there is no growth of bacteria or fungi by subjecting a part of the dermal matrix to bacterial and fungal culture. Furthermore, it is confirmed by a pathological test involving hematoxylin eosin staining that there is no abnormality in the dermal collagen structure and that the dermis is completely acellular.

1. A method for decellularizing separated skin, the method comprising treating simultaneously with a protease and a surfactant.

2. The method according to claim 1 wherein the protease is trypsin.

3. The method according to claim 1 wherein the surfactant is a polyoxyethylene p-t-octylphenyl ether surfactant.

4. An acellular dermal matrix decellularized by treating simultaneously with a protease and a surfactant.

5. The acellular dermal matrix according to claim 4 wherein the protease is trypsin.

6. The acellular dermal matrix according to claim 4 wherein the surfactant is a polyoxyethylene p-t-octylphenyl ether surfactant.

7. The acellular dermal matrix according to claim 4 wherein human allogeneic skin is used as a starting material.

8. The acellular dermal matrix according to claim 4 wherein porcine skin is used as a starting material.

9. A method for producing an acellular dermal matrix, the method comprising a decellularizing step of treating separated skin simultaneously with a protease and a surfactant.

10. The method according to claim 9 wherein the protease is trypsin.

11. The method according to claim 9 wherein the surfactant is a polyoxyethylene p-t-octylphenyl ether surfactant.

12. The method according to claim 9 wherein human allogeneic skin is used as a starting material.

13. The method according to claim 9 wherein porcine skin is used as a starting material.

14. A composite cultured skin employing as a substrate the acellular dermal matrix according to claim 4.
